(19) United States
(12) Patent Application Publication
(10) Pub. No.: US 2012/0035060 A1
(43) Pub. Date: Feb. 9, 2012

(54) HIGHLY MULTIPLEXED GENOTYPING USING LEUKOREDUCED BLOOD SAMPLES

(75) Inventors: Xin Lin, Belle Mead, NJ (US); Ermelina G. Enriquez, Plainsboro, NJ (US)

(73) Assignee: BioArray Solutions, Ltd., Warren, NJ (US)

(21) Appl. No.: 13/197,448

(22) Filed: Aug. 3, 2011

Related U.S. Application Data
(60) Provisional application No. 61/370,241, filed on Aug. 3, 2010.

(51) Int. Cl. C12Q 1/68; C07H 21/04; C07H 1/08; C40B 20/00

(52) U.S. Cl. 506/2; 435/6.11; 536/25.4

(57) ABSTRACT

Described herein are methods and kits useful for the extraction and analysis of genomic DNA from leukoreduced blood or plasma samples.

### FIELD OF THE INVENTION

The invention relates to a method for separating genomic DNA from a leukoreduced blood sample for molecular biological analysis. The invention also relates to kits for extracting DNA, analyzing DNA and removing impurities from extracted DNA.

### BACKGROUND OF THE INVENTION

Whole blood is often the least expensive and readily accessible source for genomic DNA and has the additional advantage of providing immediate visual evidence that a sample of adequate size has been obtained. However, isolating DNA from both fresh and frozen blood is difficult since only approximately 0.1% of blood cells are nucleated white blood cells (4-10×10⁷/ml). For example, 1 μl of lysed human blood contains ˜35-50 ng DNA amid ˜150 ug of protein, lipids and other components.

A variety of techniques have been developed (see FIG. 1) to isolate DNA from this complex mixture. For example in the most rigorous protocols, several milliliters of whole blood are drawn and then centrifuged to separate blood into plasma, a white blood cell (WBC) rich fraction (buffy-coat) and red blood cell (RBC) rich fraction. The WBC's are first isolated and the DNA is released using detergent lysis, followed by protease treatment and DNA purification using phenol-chloroform extraction followed by ethanol or isopropanol precipitation of the DNA The simplest reported method of DNA extraction involves boiling 1-3 μl of blood in 50 μl of water for cell lysis and directly using a portion of the lysate for further analysis. The most popular and versatile among the currently available methods are the solid phase based separation methods. In these methods whole blood is lysed in presence of an appropriate buffer that allows the released DNA to selectively adsorb on a given solid phase. This is followed by a wash step which selectively washes away the non-specifically adsorbed components, leaving the adsorbed DNA. Finally the adsorbed DNA is eluted using an appropriate elution buffer.

No matter the nature of the protocol, the recovery efficiency and the final yield of the DNA is critically dependent on the presence of a sufficient number of nucleated cells in the initial blood sample. None of the known methods explicitly address the problem of dealing with leukodepleted blood samples.

Leukodepletion is a process by which leukocytes (WBC) are removed from donated blood. It is now well established that a vast majority of febrile nonhemolytic adverse transfusion reactions are mediated by donor leukocytes. The use of leukoreduced products is thus indicated in case of the multi-transfused patients, patients receiving chemotherapy, patients undergoing bone marrow, renal or peripheral blood progenitor cell transplant and patients with hematologic malignancies. Current standards also require that, at a minimum, blood selected for transfusion to a patient be checked (phenotyped) to be antigen negative to the existing alloantibodies in the patient's serum. Recently DNA analysis has emerged as a powerful, versatile and cost effective method for blood group antigen phenotype determination. DNA analysis using blood relies on the fact that only WBCs in blood have genomic DNA. When the starting WBC concentration in whole blood is very low (as in the case of leukodepleted samples), it is difficult to carry out DNA based assays using standard DNA extraction protocols. Highly sensitive quantitative PCR techniques have been reported for quantitation of residual WBCs in filtered blood component. The analyzed samples were spiked genomic DNA cell lysate into a diluent and spiked WBCs into twice filtered fresh whole blood. A sensitivity of detection for 0.008 WBC/μl was reported. However, such assay techniques are very sophisticated, use dedicated and expensive instrumentation and typically cannot be multiplexed. At present, there is no established method or commercially available kit that can utilize leukodepleted blood as a source of genomic DNA for performing highly multiplexed genotyping assays.

A method for utilizing spent leukodepletion filter devices as a source material for the isolation and analysis of genomic DNA has been reported. However, the leukodepletion is routinely carried out only in larger blood centers and, hence, such leukocyte loaded filter devices are only available at a limited number of facilities. In most places leukodepleted donated blood is stored in a soft plastic blood collection bag. Because of potential contamination of the blood that may occur from contact with a syringe or pipette used to withdraw a sample, the blood collection bag is connected to a flexible plastic tube that is heat sealed into a series of segments containing the donor's blood. These sealed tube segments are commonly referred to as segment tubes, pigtails, or segments. The segment tubes remain attached to the blood collection bag, and are often folded into a group held together with a rubber band. Whenever the blood is to be tested, the laboratory technician simply removes one or more of the segment tubes attached to the blood collection bag for testing. Since the volume of leukodepleted blood available from the segments is limited such segment samples cannot utilized for extracting genomic DNA using the filtration device based recovery process. It has been estimated that the average content of WBCs in donated human whole blood is 10⁹/unit. By the current US standards, the total content of WBCs in a leukodepleted blood unit should be less than 5×10⁶/unit or ˜10 WBCs/μl. While it is intuitively clear, that, theoretically starting with a 3 log higher volume of blood one can compensate for the leukodepletion, for reasons discussed above, this is not a practically feasible option.

Thus, known methods for extraction and analysis of DNA from filtered or leukoreduced blood have focused on using the residual WBCs as a source of the DNA.

It has been reported that normal human plasma or serum contain small amounts of DNA that may be suitable for genetic analysis. However, it is known that due to very poor quality the analyses of such DNA are fraught with difficulties such a signal dropouts and genotyping error.

### SUMMARY OF THE INVENTION

Described herein are materials and methods for genetic analysis of mammals, for example, humans. In one embodiment, genetic analysis is performed using leukoreduced blood as a source for genomic material. Prior to the present invention, it was believed that the leukoreduced blood did not contain enough genetic material to permit analysis. The present inventors unexpectedly found that leukoreduced blood can provide enough DNA to permit genetic analysis, for example, genotyping of blood cell antigens.

In some embodiments, methods described herein typically include purifying DNA from leukoreduced blood. The DNA may be purified directly from the leukoreduced blood or the blood may be processed to remove red blood cells prior to DNA isolation. DNA isolation may be performed by any suitable technique known to those skilled in the art. Commercially available purification kits may be used, including, but not limited to those from Qiagen®, QIAamp Blood Mini Kit (Cat#51104, Qiagen, Valencia), QIAamp DNA Midi kit (cat#511850, Qiagen, Valencia, Calif.), QIAamp DSP Virus Spin Kit (Cat#61704, Qiagen, Valencia, Calif.) and QIAamp circulating nucleic acid Kit (Cat#55114, Qiagen, Valencia, Calif.).

In one embodiment whole leukodepleted blood sample is used as a source for isolation of genomic DNA.

In another embodiment the leukodepleted blood sample is first centrifuged to separate the cellular phase from plasma. Next a WBC rich plasma fraction is removed from the centrifuged blood and used as the source for isolation of genomic DNA for analysis.

In yet another embodiment the leukodepleted blood sample is first centrifuged to separate the cellular phase from plasma. Next a clear plasma fraction is removed from the centrifuged blood and used as the source for isolation of genomic DNA for analysis.

In other embodiments, described herein are kits for DNA extraction, comprising the materials required to isolate the genomic DNA from whole leukodepleted blood and WBC-rich plasma.

In yet other embodiments, described herein are kits for the analysis of extracted DNA, comprising of materials required to amplify and detect DNA.

In some embodiments, the kits described herein may include one or more of the following components: an allele specific identification primer which may include a sequence specific region, a bead capture tag and a capture probe elongation template; and a plurality of capture beads having bead capture probes attached thereto. In use, the allele specific identification primer may be used to generate single stranded DNA incorporating a labeled nucleotide, the single stranded DNA may be captured by elongation of the capture probes and elongation of the capture probe elongation template may stabilize the captured products and the capture probe elongation template does not incorporate any label carried over from the allele identification.

In another embodiment the kits described herein may include one or more of the following components: a set of allele specific identification probes which may include a sequence specific region and which may be attached to a plurality of capture beads. In use, a PCR reaction followed by post PCR enzymatic treatment may be used to generate single stranded DNA amplicons containing the polymorphisms of interest. These amplicons may be captured by the identification probes attached to the capture beads. The elongation of the capture probes incorporating labeled dNTP's indicates the presence of the corresponding polymorphism. A mismatch between the capture probe and the captured target results in no elongation and hence label incorporation.

### BRIEF DESCRIPTION OF THE DRAWINGS

FIG. 1 is a summary of genomic DNA extraction techniques

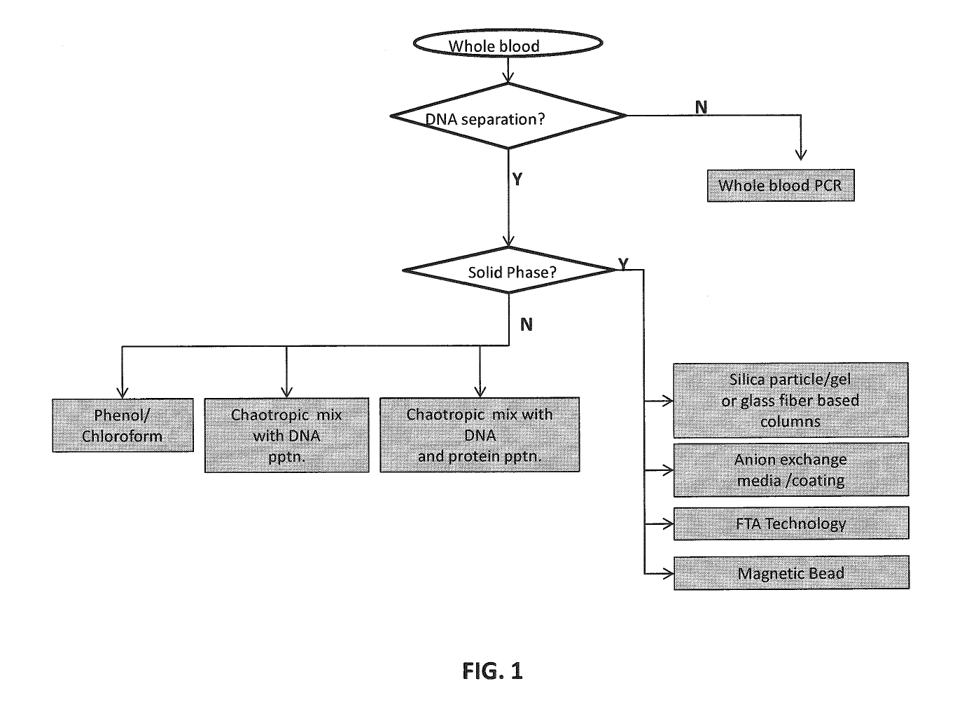

FIG. 2(a) is a diagram representing the structure of an in-solution allele identification primer. The sequence specific region, capture probe elongation template and bead capture tag are indicated.

FIG. 2(b) is a diagram representing the procedure to identify SNPs using an in-solution allele identification primer. The primer is extended only if the SNP is present and integrating a labeled nucleotide accordingly.

FIG. 2(c) is a diagram representing the bead capture of a labeled PCR extended SNP identification Primer Product. The extension from the bead using the capture probe elongation template is also indicated.

FIG. 3 is a flow chart of the Elongation Mediated Multiplexed Analysis of Polymorphisms in Solution (eMAP-S) technology.

FIG. 4 is a photograph showing a 2% agarose gel analysis of genomic DNA isolated from whole blood and LR blood. LR DNA was extracted from 400 μl of LR plasma by using QIAamp DSP virus spin kit, and WB DNA was extracted from 200 μl of whole blood by using QIAamp DNA blood Mini kit. Lane S: 100 bp standard, Lane S1-S6: WB DNA extracted from donor 1-6, Lane AS1-AS6: LR DNA extracted from donor 1-6.

FIG. 5 is a table of assay call charts before and after leukoreduction for Example 4

FIG. 6 is a table of assay call charts before and after leukoreduction for Example 5.

FIG. 7 is a table of assay call charts before and after leukoreduction for Example 6.

### DETAILED DESCRIPTION OF INVENTION

Disclosed herein are methods and reagents, including, but not limited to kits, to separating genomic DNA from a leukoreduced blood sample for molecular biological analysis. Further disclosed are kits for extracting DNA, analyzing DNA and removing impurities from extracted DNA.

“Leukodepletion” as used herein refers to processes by which leukocytes (WBC) are removed from donated blood.

“Leukodepleted” as used herein refers to a sample of donated blood that has undergone a process to have the leukocytes removed. Also, the terms “leukodepleted” and “leukoreduced” are used interchangeably herein.

Whole Blood Samples

As discussed above, blood samples from a donor will contain many leukocytes, and harbor genomic DNA. In an effort to deplete the blood sample of leukocytes, a number of approaches may be used. In some embodiments, whole blood may be separated into fractions using routine procedures known in the art, including preparation of leukocyte samples, buffy coats, plasma samples, and RBC enriched samples. Such procedures generally include treatment with ficoll, percoll, or another type of sedimentation agent to separate the various blood fractions. In other embodiments, the whole blood sample may be first subjected to a leukodepletion protocol as referred to above. Alternatively, methods may begin using a leukodepleted blood sample. If that is the case, centrifugation generally will remove any residual leukocytes and fractionate the sample into a pure plasma region, an enriched WBC/plasma interface and a RBC rich region in the bottom of the tube. The pure plasma region could be used for analysis. Alternatively, the plasma region is removed and subjected to another centrifugation to concentrate any residual WBCs. The clarified plasma region could also be used for analysis.

In some embodiments, the sample used for analysis is substantially free of leukocytes or WBCs. Alternatively, the content of WBCs in a leukodepleted blood unit used for analysis should be less than 5×10⁶/unit or ˜10 WBCs/μl.

DNA Preparation from Leukodepleted Samples

DNA preparation or extraction from samples for analysis may be performed using routine methods known in the art. Alternatively, DNA purification kits, for purifying DNA from various sources may be employed to extract DNA from leukodepleted samples. Accordingly, DNA kits and protocols, including, but not limited to the kits listed below may be employed in the methods herein.

In some embodiments, methods of the invention comprise extraction of DNA from WBC rich plasma using QIAamp DNA Mini Blood kit. The protocol for extracting DNA from WBC rich plasma is reproduced below.

Reagent Preparation

Prepare QIAamp DNA Mini Blood kit reagents as indicated in the product manual (Protease Solution, Buffer AW1, Buffer AW2). Equilibrate all reagents to room temperature.

A protocol for extracting DNA from leukoreduced segments is described below:

Procure three or four segments of leukoreduced blood approximately 2 ml or more for the samples to be tested. Label a 2.0 mL centrifuge tube with the sample ID cut the ends of the blood tubing and carefully transfer the leukoreduced blood into the 2.0 ml micro-centrifuge tube. Prepare WBC rich plasma by centrifuging blood (˜2 ml obtained from 3-4 LR segments) at 2500×g for 10 minutes at room temperature. After centrifugation, three different fractions are distinguishable: the upper clear layer is pure plasma; the intermediate layer WBC rich plasma, containing concentrated leukocytes; and bottom layer contains concentrated erythrocytes (Red blood cells). Discard the upper clear layer, and remove 200 μl of the intermediate layer (WBC rich plasma).

QIAcube

Label one 2.0 ml tube for initial sample collections. Add exactly the volume of Protease Solution described in the protocol sheet (supplied by the manufacturer) per samples in 1.5 ml tube and transfer 200 μl of WBC rich plasma sample into the labeled 2.0 ml tubes. Turn Qiacube power on and follow and confirm step by step prompts. When protocol is finished, retain the 1.5 ml tube with eluate and discard the remaining consumables. Extracted LR DNA is ready for PCR. Store leukoreduced DNA at −20° C. or below (defrost-free) until use. Avoid multiple freeze/thaw cycles.

In some embodiments, methods described herein comprise extraction of DNA from leukoreduced whole blood sample using the QIAAmp DNA Midi kit. Another protocol for extracting DNA from leukoreduced segments is listed below:

Reagent Preparation

Prepare QIAamp DNA Midi Blood kit reagents as indicated in the product manual (Protease Solution, Buffer AW1, Buffer AW2, Buffer AE). Equilibrate all reagents to room temperature.

Leukoreduced Sample

Procure three or four segments of leukocyte reduced blood approximately 2 ml or more.

Protocol:

Equilibrate all reagents and samples to room temperature (15-25° C.) before starting. Pipet 200 μl QIAGEN Protease into the bottom of a 15 ml centrifuge tube. If the sample volume is less than 2 ml, reduce the amount of QIAGEN Protease. Add 2 ml blood and mix briefly. Add 2.4 ml Buffer AL and mix thoroughly by inverting the tube 15 times, followed by additional vigorous shaking for at least 1 min. Incubate at 70° C. for 10 min. Add 2 ml ethanol (96-100%) to the sample and mix again by vortexing. Carefully transfer all or one half of the solution (3.3 ml) from step 5 onto the QIAamp Midi column placed in a 15 ml centrifugation tube. Close the cap and centrifuge at 1850×g (3000 rpm) for 3 min. Remove the QIAamp Midi column, discard the filtrate, and place the QIAamp Midi column back into the 15 ml centrifugation tube. Load the remainder of the solution from step 6 onto the QIAamp Midi column Close the cap and recentrifuge at 1850×g (3000 rpm) for 3 min. Remove the QIAamp Midi column, discard the filtrate, and place the QIAamp Midi column back into the 15 ml centrifugation tube. Carefully, without moistening the rim, add 2 ml Buffer AW1 to the QIAamp Midi column. Close the cap and centrifuge at 4500×g (5000 rpm) for 1 min. Carefully, without moistening the rim, add 2 ml of Buffer AW2 to the QIAamp Midi column Close the cap and centrifuge at 4500×g (5000 rpm) for 15 min. Place the QIAamp Midi column in a clean 15 ml centrifugation tube (provided), and Add 100 μl of Buffer AE, equilibrated to room temperature Pipet directly onto the membrane of the QIAamp Midi column and close the cap. Incubate at room temperature for 5 min and centrifuge at 4500×g (5000 rpm) for 5 min. Extracted LR DNA is ready for PCR. For long term storage of DNA, elute in Buffer AE and storing in aliquots at −20° C. Avoid multiple freeze/thaw cycles.

In some embodiments, methods described herein comprise extraction of DNA from plasma using the QIAamp DSP Virus Spin Kit. Another DNA extraction protocol is listed below:

Reagent Preparation

QIAamp DSP Virus Spin Kit (Cat#61704, Qiagen, Valencia, Calif.) reagents were prepared as indicated in the product manual. Namely, Protease Solution, Buffer AW1 and Buffer AW2. All reagents were allowed to equilibrate to room temperature.

Leukoreduced Segments

Three or four segments of leukoreduced blood, totaling to about 2 ml or more by volume was suitable as samples to be tested. A 2.0 ml centrifuge tube was labeled with the sample ID. The ends of the segment blood tubing were cut and the contents carefully transferred into the 2.0 ml micro-centrifuge tube. Plasma was by prepared by centrifuging blood (−2 ml obtained from 3-4 LR segments) at ˜2500×g for 10 minutes at room temperature. After centrifugation, two different fractions are distinguishable: the upper clear layer is pure plasma and bottom layer contains concentrated cells (erythrocytes and residual leukocytes). The upper pure plasma layer (˜1 ml) was aspirated and moved into new a tube.

QIAcube Extraction

A 2.0 ml tube was labeled and required volume of Protease Solution, AVE buffer, and carrier RNA added to it as described in the protocol sheet supplied by the manufacturer. Following this, 400 μl of pure plasma sample was transferred into the tube. Using the sample the QIAamp DSP virus larger volume standard protocol was then run on The QIAcube instrument following and confirming step-by-step prompts. Extracted LR DNA obtained at the completion of the QIAcube run is ready for PCR. The extracted leukoreduced DNA was stored at −20° C. or below (defrost-free) until use.

In some embodiments, methods of the invention comprise extracting DNA from plasma using the QIAamp Circulating Nucleic Acid Kit. In other embodiments, methods of the invention comprise extraction of DNA from leukoreduced whole blood sample using the QIAamp Circulating Nucleic Acid Kit. Another DNA extraction procedure is listed below.

Reagent Preparation

QIAamp circulating nucleic acid Kit (Cat#55114, Qiagen, Valencia, Calif.) reagents were prepared as indicated in the product manual. Namely, Buffer ACW1, Buffer ACW2 and lysis buffer with of carrier RNA. All reagents were allowed to equilibrate to room temperature.

Plasma Preparation and Manual Procedure

A 15 ml centrifuge tube was labeled with the sample ID and ˜6 ml of leukoreduced blood was transferred into the tube. Plasma was prepared by centrifuging 6 mL of LR blood at 2500×g for 10 minutes at room temperature. After centrifugation, two different fractions are distinguishable: the upper clear layer is pure plasma and bottom layer contains concentrated cells (erythrocytes and residual leukocytes). The upper pure plasma layer (˜3 ml) was removed into a new tube and the rest discarded. Next, about 300 μl of the QIAGEN Proteinase K reagent was added to a 15 ml centrifuge tube, followed by addition of 3 ml plasma and 2.4 ml Buffer ACL. The contents of the tube were mixed thoroughly by inverting the tube 4 times, followed by additional vigorous shaking for at least 1 min. The tube with its contents was then allowed to incubate at 60° C. for 30 min. Next, 5.4 ml Buffer ACB was added to the tube and the contents mixed again by vortexing followed by incubation on ice for ˜5 min. The tube contents (lysate) were then carefully transferred to the tube-extender of QIAamp Mini column. The lysate was pulled through the column by the vacuum aspiration. The QIAamp Mini column was then removed and placed in the QIAcube instrument.

Automated Procedure on QIAcube

A 1.5 ml tube was labeled for collection of the eluted g-DNA. In a 2 ml tube without skirt (supplied with kit), 100 μl of AVE buffer described in the protocol sheet (supplied by the manufacturer) was added. Next, the 2.0 ml tube, the labeled 1.5 ml elution tube and the QIAamp Mini column were placed at their designated positions in the QIAcube. The QIAamp circulating nucleic acid standard protocol was then run on the QIAcube instrument, following and confirming step by step prompts. Extracted LR DNA obtained at the completion of the QIAcube run is ready for PCR. The extracted leukoreduced DNA was stored at −20° C. or below (defrost-free) until use.

In yet other embodiments, extracted DNA is analyzed through multiplexed genotyping using an HEA LR eMAP-S BeadChip kit.

In one embodiment the extracted genomic DNA is analyzed for single nucleotide polymorphisms (SNPs) associated with blood group systems using an HEA BeadChip kit (BioArray Solutions, Ltd., Warren, N.J.). The data is acquired using an AIS 400 instrument and the analysis carried out using HEA Analysis Software package in the BioArray Solutions Information System (BASIS) (BioArray Solutions, Ltd., Warren, N.J.).

Twenty-four polymorphisms associated with thirty Human Erythrocyte Antigens and one with hemoglobinopathies included in the BioArray Solutions HEA LR eMAP-S BeadChip Kit are listed below.

TABLE 1
Selected Polymorphisms Associated with Human
Erythrocyte Antigens and Hemoglobinopathies

| Blood Factor | Analyte | Polymorphism |
|---|---|---|
| Rh | C/c | 307C > T |
| | | 109 lns |
| | E/e | 676G > C |
| | VS | 733C > G |
| | V | 1006G > T |
| Kell | K/k | 698T > C |
| | Js^{a}/Js^{b} | 1910C > T |
| | Kp^{a}/Kp^{b} | 961C > T |
| Duffy | Fy^{a}/Fy^{b} | 125G > A |
| | GATA (Silencing FY) | −33T > C |
| | Fy^{x}[Fy(b+^{w})] | 265C > T |
| Kidd | Jk^{a}/Jk^{b} | 838G > A |
| MNS | M/N | 59C > T |
| | S/s | 143T > C |
| | Silencing S | Ex5 230C > T |
| | Silencing S | In5 G > T |
| Lutheran | Lu^{a}/Lu^{b} | 230A > G |
| Dombrock | Do^{a}/Do^{b} | 793A > G |
| | Hy+/Hy− | 323G > T |
| | Jo(a+)/Jo(a−) | 350C > T |
| Cartwright | YT^{a}/YT^{b} | 1057 C > A |
| Diego | Di^{a}/Di^{b} | 2561T > C |
| Colton | Co^{a}/Co^{b} | 134C > T |
| Hemoglobin S | HgbS | 173A > T |

In some embodiments, methods of the invention comprise analyzing at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23 or more polymorphisms listed in Table 1. In other embodiments, methods of the invention comprise analyzing 1, 2, 3, 4, 5, 6, 7, 8, 9 10, 11, 12, 13, 14, 15, 16, 17, 18, 19, 20, 21, 22, 23, or 24 polymorphisms listed in Table 1.

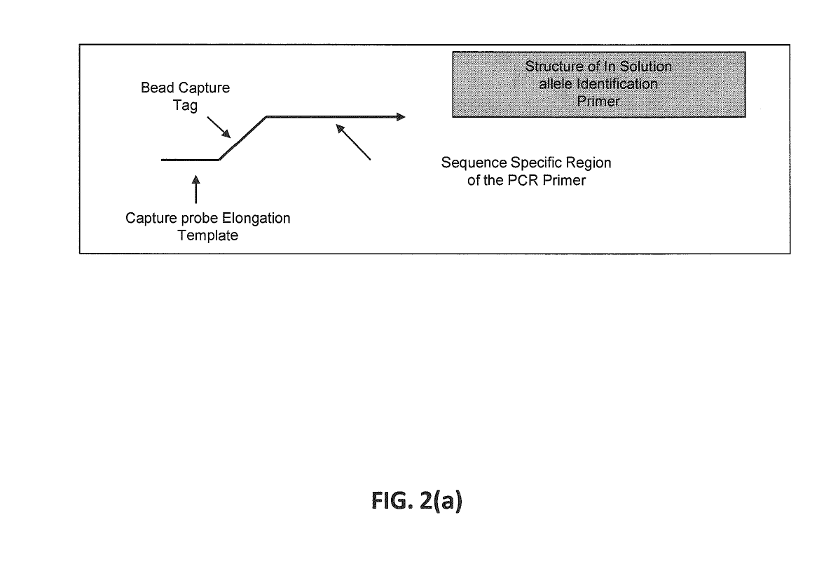

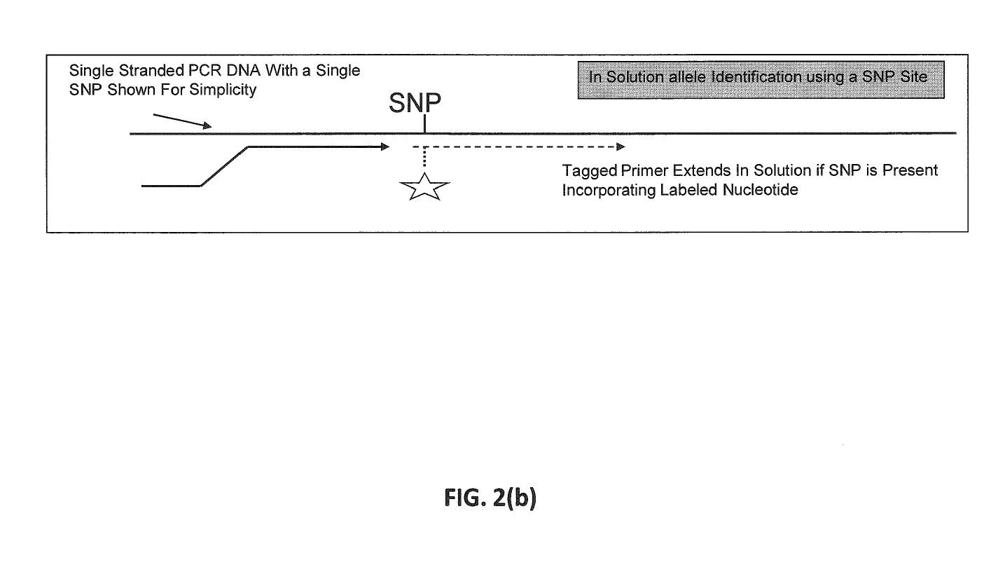

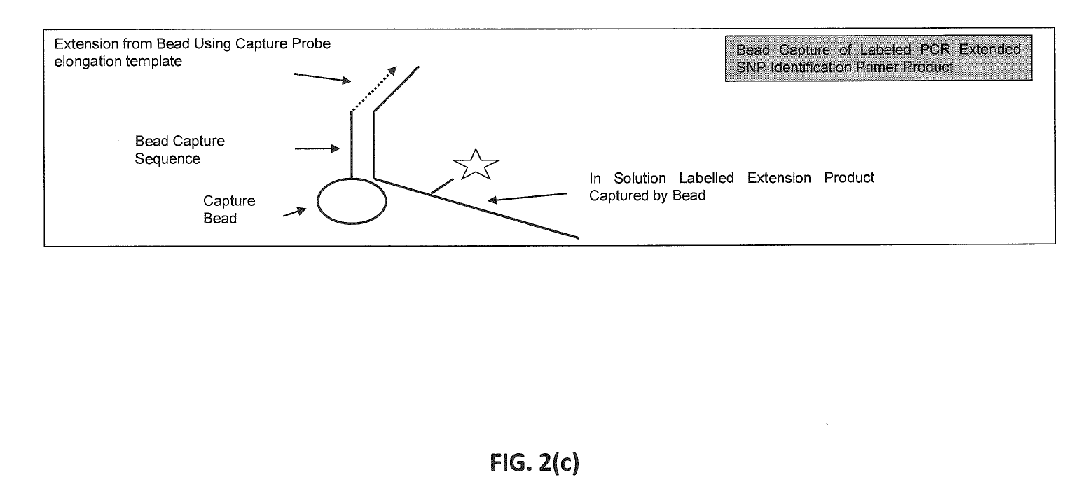

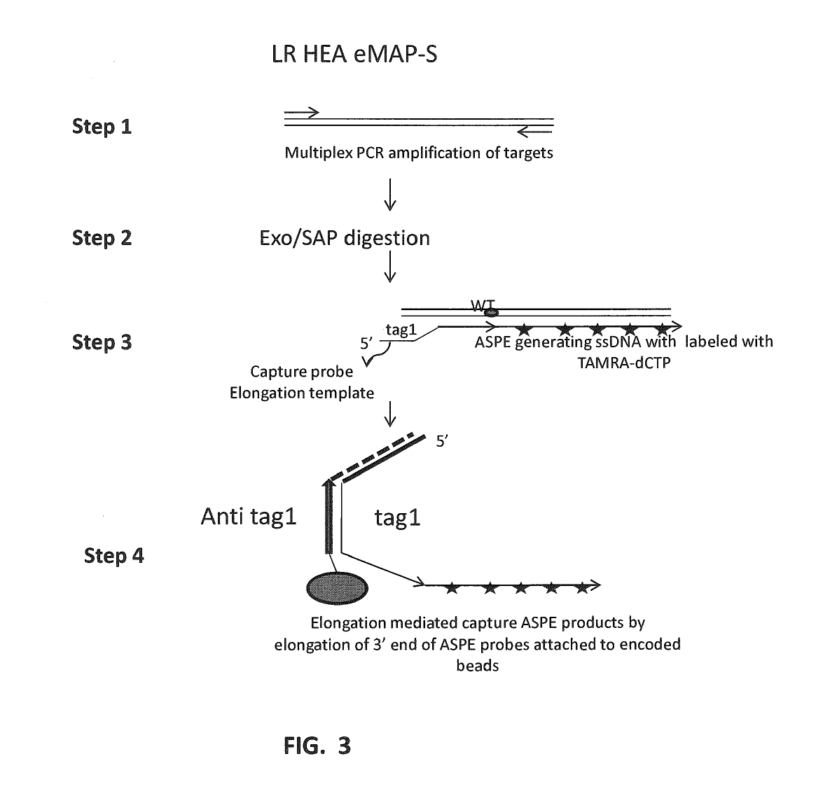

The BioArray Solutions HEA LR eMAP-S BeadChip Kit uses the novel Elongation Mediated Multiplexed Analysis of Polymorphisms in Solution (eMAP-S) technology (see FIG. 3) to identify the presence or absence of the selected alleles associated with a given phenotype. Multiplex polymerase chain reaction (PCR) is used to generate multiple copies of the DNA region containing information for the antigens of interest. After multiplex PCR amplification, a post-PCR clean-up step is done to remove dNTPs (nucleotide triphosphates) and excess primers. An allele specific primer extension (ASPE) PCR is used to generate single stranded DNA with fluorescently-labeled molecules along with a 5′ capture “tag” sequence in solution. The single stranded DNAs from ASPE product are subsequently captured by elongation of capture probe on the BeadChip™. To stabilize the duplex products formed by capture of ASPE products to bead-displayed oligonucleotide capture probes, said probes are permitted to undergo elongation, initiated at the 3′ end of the oligonucleotide immobilized on the surface of the bead, using the Capture Probe Elongation Template, as shown in FIG. 2(c). The Capture Probe Elongation Template of the ASPE primers does not allow the incorporation of any label carried over from the earlier in solution allele identification. For example if the assay used labeled dCTP in the ASPE step (see FIG. 2(b)) the DNA sequence of the Capture Probe Elongation Template of the in-solution allele identification Primers (see FIG. 2 (a)) would not contain any G residues.

In other embodiments, methods of the invention comprise multiplexed genotyping using an HEA LR eMAP BeadChip kit.

In another embodiment the extracted genomic DNA may be analyzed for single nucleotide polymorphisms (SNPs) associated with blood group systems using HEA LR eMAP BeadChip kit (BioArray Solutions, Ltd., Warren, N.J.). The data is acquired using an AIS 400 instrument and the analysis carried out using HEA Analysis Software package in the BioArray Solutions Information System (BASIS) (BioArray Solutions, Ltd., Warren, N.J.).

The BioArray Solutions HEA LR eMAP BeadChip Kit uses the novel Elongation Mediated Multiplexed Analysis of Polymorphisms (eMAP) technology to identify the presence or absence of the selected alleles associated with a given phenotype. Multiplex polymerase chain reaction (PCR) is used to generate multiple copies of the DNA region containing information for the antigens of interest. After multiplex PCR amplification, a post-PCR clean-up step is done to remove dNTPs (nucleotide triphosphates) and excess primers. Following, an enzymatic digestion using the enzyme Lambda Exonuclease is carried out in order to generate single stranded amplicons. The single stranded DNAs from post-PCR step are subsequently captured using allele specific capture probes on the BeadChip™. The allele specific capture probes contain variable 3′ termini matching the normal or a variant allele. In the event of a match between the 3′ termini of the probe and the captured target, the probes undergo elongation, initiated at the 3′ end. During elongation labeled dNTP's get incorporated into the elongated strand. Each bead produces an assay signal reflecting the incorporation of labeled dNTP's into the elongation products displayed on that bead.

Elongation products of the variant and normal sequences are simultaneously detected by instant imaging of the entire array using the AIS-400 Array Imaging System. In this method, each capture probe is covalently attached to a spectrally distinguishable bead type. A library of individual bead types contains all of the probes of interest, including internal positive, negative, and system controls. The library is immobilized in the BeadChip™ array, allowing for the simultaneous detection of the polymorphisms of interest. The BioArray Solutions Array Imaging System (AIS 400) is used to capture the fluorescent signal from individual beads in an image of the array, determine the identity of the bead by its position in the array, and report the average signal intensity, coefficient of variance of the intensities, and number of beads measured for each type of probe. The HEA Analysis Software in BioArray Solutions Information System (BASIS) imports the raw intensity output, assesses the validity of the internal controls, and generates assay results. This is a qualitative test. However, if the signal intensity for any specific allele is too low (Low Signal, LS), the genotype assignment for that allele cannot be successfully completed. Thus the presence or the number of LS calls can be used to judge the quality of any particular assay run. LS calls can be triggered by not having target DNA present in sufficient quantity. It is worthwhile to mention that such LS calls can also be a result of polymerase chain reaction (PCR) failure, due to the presence of PCR impurities in the extracted genomic DNA. Many such inhibitory substances are inherent to whole blood samples. One attribute of the instant invention is that it effectively eliminates the introduction of sources of impurities to the genomic DNA extraction step.

### Specific Embodiments

1. A method for genetic analysis, comprising: isolating DNA from a sample comprising leukoreduced blood; and analyzing the DNA for the presence of one or more genetic markers.

2. The method of embodiment 1, wherein the sample comprises red blood cells.

3. The method of embodiment 1, wherein the step of isolating DNA comprises removing red blood cells from the sample prior to isolation of the DNA.

4. The method of embodiment 1, wherein the step of analyzing DNA comprises determining the presence or absence of one or more alleles of interest.

5. The method of embodiment 4, wherein the one or more alleles are polymorphisms in one or more human erythrocyte antigens.

6. The method of embodiment 5, wherein the antigens are selected from the group consisting of Rh, Kell, Duffy, Kidd, MNS, Lutheran, Dombrock, Cartwright, Diego, Colton, and Hemoglobin S.

7. The method of embodiment 1, wherein said leukoreduced blood contains less than 10 leukocytes/μl of sample.

8. The method of embodiment 1, wherein said DNA is isolated from a sample present on a silica based membrane.

9. The method of embodiment 1, wherein said DNA is amplified prior to analysis.

10. The method of embodiment 1, wherein said DNA is isolated using magnetic beads.

11. The method of embodiment 1, wherein said DNA exhibits an A260/280 ratio of 1.5 or higher.

12. The method of embodiment 1, wherein said method isolates more than 50 ng DNA/ml of sample.

13. The method of embodiment 1, wherein said isolated DNA is subjected to multiplexed genotyping.

14. The method of embodiment 1, wherein the DNA is analyzed using an allele specific identification primer having a sequence specific region, a bead capture tag and a capture probe elongation template.

15. The method of embodiment 14, wherein the method comprises annealing a DNA to the allele specific identification primer.

16. The method of embodiment 15, wherein the step of annealing facilitates production of a single stranded DNA primed from the allele specific identification primer.

17. The method of embodiment 16, wherein said single-stranded DNA incorporates a labeled nucleotide.

18. The method of embodiment 1, wherein the DNA is analyzed using a plurality of capture beads.

19. The method of embodiment 18, wherein the plurality comprises beads specific for different targets.

20. The method of any of embodiments 14-19, wherein the beads are immobilized on an array.

21. The method of embodiment 1, wherein the step of isolation of DNA employs a kit selected from the group consisting of QIAamp Blood Mini Kit, QIAamp DNA Midi kit, QIAamp DSP Virus Spin Kit and QIAamp circulating nucleic acid Kit.

22. The method of embodiment 1, wherein the step of analyzing the DNA employs a kit selected from the group consisting of: HEA LR eMAP-S BeadChip Kit and HEA LR eMAP BeadChip Kit.

23. A kit for analyzing extracted DNA comprising: an allele specific identification primer having a sequence specific region, a bead capture tag and a capture probe elongation template; a plurality of capture beads having bead capture probes attached thereto; and a container therefore.

24. The kit of embodiment 23, wherein the allele specific identification primer generates single stranded DNA from a target sequence in the extracted DNA incorporating a labeled nucleotide, the single stranded DNA is captured by elongation of the bead capture probes and the capture probe elongation template does not incorporate the label from the allele identification.

25. A method of isolating DNA from a leukoreduced blood sample, or plasma sample, comprising:
a. Providing a leukoreduced blood sample or plasma sample; and
b. Isolating more than 50 ng DNA/ml of sample.

26. The method of embodiment 25, wherein said leukoreduced blood contains less than 10 leukocytes/μl of sample.

27. The method of embodiment 25, wherein said DNA is isolated using magnetic beads.

28. The method of embodiment 25, wherein said DNA exhibits an A260/280 ratio of 1.5 or higher.

29. The method of embodiment 25, wherein said DNA is suitable for multiplexed genetic analysis.

30. A method of isolating genomic DNA from a blood sample comprising:
a. Providing a leukoreduced blood sample by removing the leukocytes from the sample; and
b. Isolating DNA from said sample by contacting gDNA to a substrate that selectively binds DNA; and
c. Eluting the DNA from said substrate wherein said method isolates more than 50 ng DNA/ml of sample.

31. The method of embodiment 30, wherein said leukoreduced blood contains less than 10 leukocytes/μl of sample.

32. The method of embodiment 30, wherein said DNA is isolated using magnetic beads.

33. The method of embodiment 30, wherein said isolated DNA exhibits an A260/280 ratio of 1.5 or higher.

34. The method of embodiment 30, wherein said isolated DNA is suitable for multiplexed genetic analysis.

### EXAMPLES

### Example 1

Cell Count Analysis of Un-Filtered and Leukoreduced Samples

10 whole blood samples were procured and taken through leukodepletion. The leukocyte count before and after filtration is shown in Table 2 below.

An additional 6 whole blood samples were procured and taken through leukodepletion. The leukocyte count before and after filtration is shown in Table 3 below.

TABLE 2
Cell counts before and after Leukodepletion

| | UN-FILTERED | | LEUKOCYTE DEPLETED | |
|---|---|---|---|---|
| | | CELL COUNT | | CELL COUNT |
| DONOR | LOT NUMBER | (CELLS/uL) | LOT NUMBER | (CELLS/uL) |
| 1 | BRH375641 | 4,059.02 | BRH375651 | 1.55 |
| 2 | BRH375642 | 4,420.88 | BRH375652 | 2.27 |
| 3 | BRH375643 | 2,308.62 | BRH375653 | 0.36 |
| 4 | BRH375644 | 5,636.80 | BRH375654 | 2.21 |
| 5 | BRH375645 | 5,508.74 | BRH375655 | 2.63 |
| 6 | BRH375646 | 6,194.51 | BRH375656 | 2.58 |
| 7 | BRH375647 | 3,261.68 | BRH375657 | 1.13 |
| 8 | BRH375648 | 4,365.56 | BRH375658 | 0.93 |
| 9 | BRH375649 | 6,052.18 | BRH375659 | 0.62 |
| 10 | BRH375650 | 5,975.22 | BRH375660 | 1.18 |

TABLE 3
Cell counts before and after Leukodepletion

| | UN-FILTERED | | LEUKOCYTE DEPLETED | |
|---|---|---|---|---|
| | | CELL COUNT | | CELL COUNT |
| DONOR | LOT NUMBER | (CELLS/uL) | LOT NUMBER | (CELLS/uL) |
| 1 | BRH462161 | 7,189.32 | BRH462167 | 1.64 |
| 2 | BRH462162 | 6,291.40 | BRH462168 | 2,857.15 |
| 3 | BRH462163 | 9,410.52 | BRH462169 | 1.89 |
| 4 | BRH462164 | 6,359.77 | BRH462170 | 1.69 |
| 5 | BRH462165 | 4,781.95 | BRH462171 | 1.28 |
| 6 | BRH462166 | 6,809.75 | BRH462172 | 0.67 |

### Example 2

Extracted DNA Concentration in Unfiltered and Leukodepleted Samples

The paired DNA samples (before and after filtration) shown in Table 2 were characterized for DNA concentration using UV-vis Spectroscopy. The results for the first eight samples are summarized in Table 4 below. The results indicate that even though the concentration of DNA is lower after filtration when compared to the concentration of DNA recovered using samples before filtration, the absolute values are well above the average concentrations reported for small amounts of DNA present in normal Human plasma.

TABLE 4
DNA concentration before and after leukodepletion

| | | ng/ml of | | | |
|---|---|---|---|---|---|
| Sample ID | ng/μL | sample | A260 | 260/280 | 260/230 |
| Donor1-WB-Qiacube | 34.39 | 573.16 | 0.688 | 1.85 | 0.99 |
| Donor1-LR-midi | 20.62 | 343.66 | 0.412 | 1.89 | 0.98 |
| Donor2-WB-Qiacube | 31.6 | 726.66 | 0.632 | 1.99 | 0.99 |
| Donor2-LR-midi | 16.98 | 283.00 | 0.34 | 1.59 | 0.87 |
| Donor3-WB-Qiacube | 20.11 | 335.16 | 0.402 | 2.02 | 0.71 |
| Donor3-LR-midi | 8 | 133.34 | 0.16 | 1.68 | 0.48 |
| Donor 4--WB-Qiacube | 47.4 | 790 | 0.948 | 1.93 | 1.11 |
| Donor 4--LR-midi | 14.07 | 234.5 | 0.281 | 2.41 | 0.77 |
| Donor 5--WB-Qiacube | 46.92 | 782 | 0.938 | 1.9 | 1.11 |
| Donor 5--LR-midi | 16.88 | 281.32 | 0.338 | 1.91 | 0.87 |
| Donor 6--WB-Qiacube | 56.49 | 941.50 | 1.13 | 1.87 | 1.21 |
| Donor 6--LR-midi | 16.31 | 271.82 | 0.326 | 2.24 | 0.85 |
| Donor 7--WB-Qiacube | 25.51 | 425.16 | 0.51 | 2.02 | 0.85 |
| Donor 7--LR-midi | 11.78 | 196.34 | 0.236 | 1.95 | 0.72 |
| Donor 8--WB-Qiacube | 35.74 | 595.66 | 0.715 | 1.87 | 0.87 |
| Donor 8--LR-midi | 8.98 | 149.66 | 0.18 | 2.76 | 0.66 |

### Example 3

Agarose Gel Analysis of Unfiltered and Leukodepleted Samples

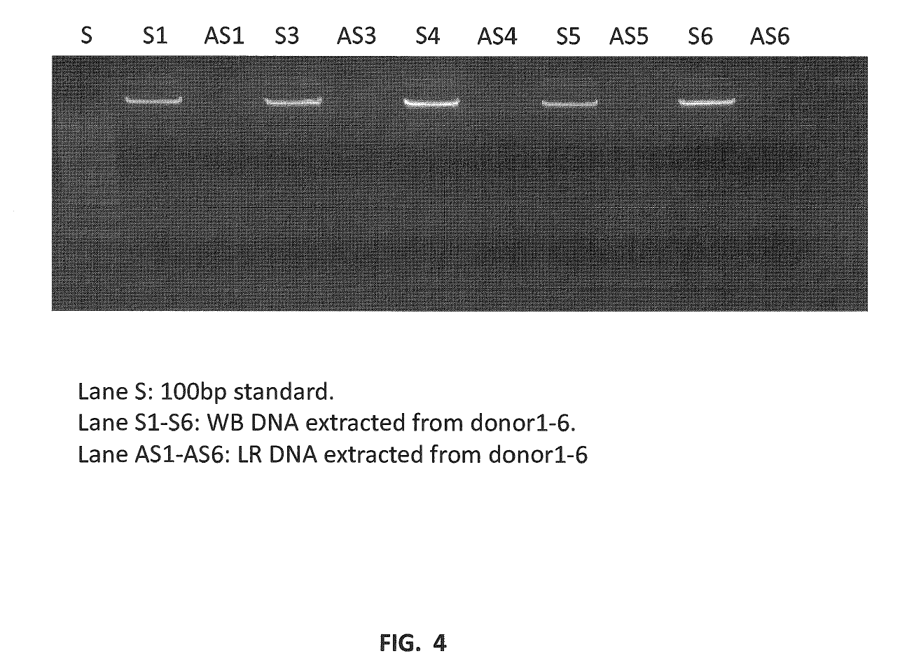

Identical samples both in the form of whole blood and corresponding leukoreduced form were procured and characterized (see Table 3). In order to generate the paired samples genomic DNA was isolated from whole blood and the corresponding LR blood. LR DNA was extracted from 400 μl of LR plasma by using the QIAamp DSP Virus Spin Kit method outlined above and WB DNA was extracted from 200 μl of whole blood by using QIAamp DNA blood Mini kit. FIG. 4 shows the results obtained from running a 2% agarose gel analysis. Results indicate absence of high molecular weight DNA in the LR samples.

### Example 4

HEA LR eMAP-S Beadchip Kit Results Using Whole and Whole Leukoreduced Blood

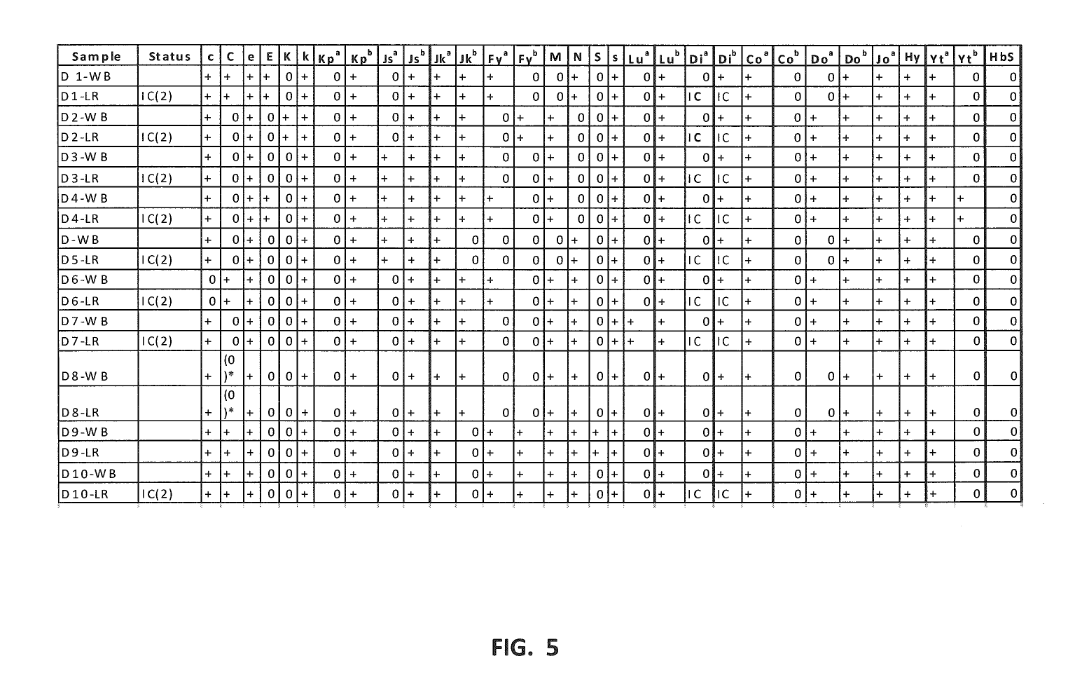

The whole blood samples were extracted using QIAamp Blood Mini Kit and the LR samples were extracted using QIAamp DNA Midi kit. The paired samples were run using the HEA LR eMAP-S BeadChip kit. The results are shown in FIG. 5. Except for the “Di” marker (where several “indeterminate call” were recorded due to low signals on this marker) the calls were all concordant.

### Example 5

HEA LR eMAP-S Beadchip Kit Results Using Whole Blood and Plasma from Leukoreduced Blood

The whole blood samples were extracted using QIAamp Blood Mini Kit and LR sample extracted using QIAamp DSP Virus Spin Kit.

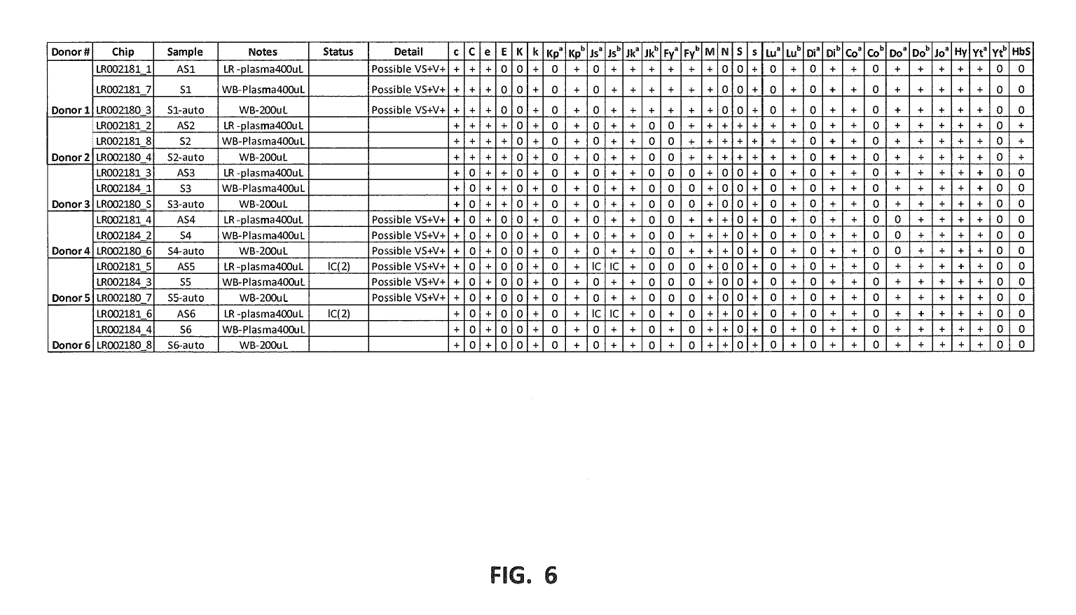

The paired samples were run using the HEA LR eMAP-S BeadChip kit. The results are shown in FIG. 6. As a comparison DNA extracted from plasma obtained from the whole blood sample was also run. No discordant calls were noted other than two incidences of signal dropouts on the “Js” probe.

### Example 6

HEA LR eMAP-S Beadchip Kit Results Using Whole Blood and Plasma from Leukoreduced Blood

The whole blood samples were extracted using QIAamp Blood Mini Kit and LR sample extracted using QIAamp circulating nucleic acid Kit.

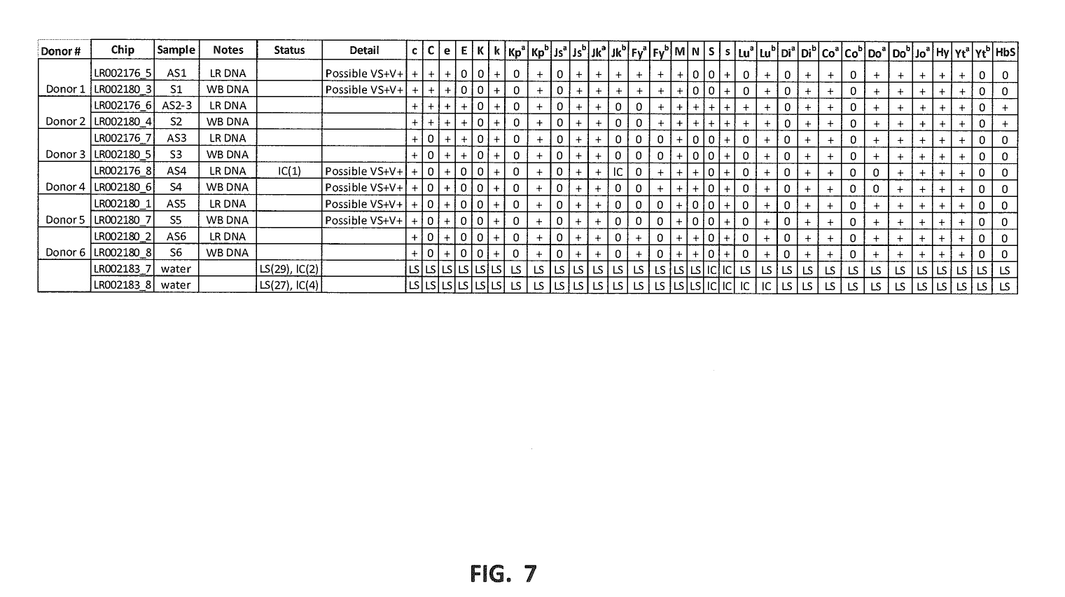

The paired samples were run using the HEA LR eMAP-S BeadChip kit. The results are shown in FIG. 7. As a no template control a sample of water was also run. No discordant calls were noted other than one incidence of signal dropout on the “Jk” probe.

While the foregoing invention has been described in some detail for purposes of clarity and understanding, it will be appreciated by one skilled in the art from a reading of this disclosure that various changes in form and detail can be made without departing from the true scope of the invention and appended claims. All patents and publications cited herein are entirely incorporated herein by reference.

1. A method for genetic analysis, comprising: isolating DNA from a sample comprising leukoreduced blood; and analyzing the DNA for the presence of one or more genetic markers.

2. (canceled)

3. The method of claim 1, wherein the step of isolating DNA comprises removing red blood cells from the sample prior to isolation of the DNA.

4. The method of claim 1, wherein the step of analyzing DNA comprises determining the presence or absence of one or more alleles of interest.

5. The method of claim 4, wherein the one or more alleles are polymorphisms in one or more human erythrocyte antigens.

6. The method of claim 5, wherein the antigens are selected from the group consisting of Rh, Kell, Duffy, Kidd, MNS, Lutheran, Dombrock, Cartwright, Diego, Colton, and Hemoglobin S.

7. The method of claim 1, wherein said leukoreduced blood contains less than 10 leukocytes/μl of sample.

8. (canceled)

9. The method of claim 1, wherein said DNA is amplified prior to analysis.

10. The method of claim 1, wherein said DNA is isolated using magnetic beads.

11. The method of claim 1, wherein said DNA exhibits an A260/280 ratio of 1.5 or higher.

12. The method of claim 1, wherein said method isolates more than 50 ng DNA/ml of sample.

13. The method of claim 1, wherein said isolated DNA is subjected to multiplexed genotyping.

14. The method of claim 1, wherein the DNA is analyzed using an allele specific identification primer having a sequence specific region, a bead capture tag and a capture probe elongation template.

15. The method of claim 14, wherein the method comprises annealing a DNA to the allele specific identification primer.

16. The method of claim 15, wherein the step of annealing facilitates production of a single stranded DNA primed from the allele specific identification primer.

17. The method of claim 16, wherein said single-stranded DNA incorporates a labeled nucleotide.

18. The method of claim 1, wherein the DNA is analyzed using a plurality of capture beads.

19. (canceled)

20. (canceled)

21. The method of claim 1, wherein the step of isolation of DNA employs a kit selected from the group consisting of QIAamp Blood Mini Kit, QIAamp DNA Midi kit, QIAamp DSP Virus Spin Kit and QIAamp circulating nucleic acid Kit.

22. The method of claim 1, wherein the step of analyzing the DNA employs a kit selected from the group consisting of: HEA LR eMAP-S BeadChip Kit and HEA LR eMAP BeadChip Kit.

23. A kit for analyzing extracted DNA comprising: an allele specific identification primer having a sequence specific region, a bead capture tag and a capture probe elongation template; a plurality of capture beads having bead capture probes attached thereto; and a container therefore.

24. (canceled)

25. A method of isolating DNA from a leukoreduced blood sample or plasma sample, comprising:
a. Providing a leukoreduced blood sample or plasma sample; and
b. Isolating more than 50 ng DNA/ml of sample.

26-34. (canceled)
